(19) United States
(12) Patent Application Publication
(10) Pub. No.: US 2005/0085705 A1
(43) Pub. Date: Apr. 21, 2005

(54) fMRI system for use in detecting neural abnormalities associated with CNS disorders and assessing the staging of such disorders

(76) Inventors: Stephen M. Rao, Shorewood, WI (US); Catherine L. Elsinger, Wauwatosa, WI (US)

Correspondence Address: John J. Horn;Patent Counsel, Neurognostics, Inc., 10437 Innovation Drive, Suite 309, Milwaukee, WI, 53226-4815, US

(21) Appl. No.: 10/970,927

(22) Filed: Oct. 21, 2004

Related U.S. Application Data
(60) Provisional application No. 60/512,940, filed on Oct. 21, 2003.

(51) Int. Cl. G06F 19/00; G01N 33/48; G01N 33/50; A61B 5/05

(52) U.S. Cl. 600/345; 702/19

(57) ABSTRACT

A system for detecting neural abnormalities associated with specific central nervous system disorders in patients suspected of suffering from said disorders and for assisting in assessing the staging of said disorders in patients suffering from said disorders. Magnetic resonance imaging (MRI) machines and functional magnetic resonance imaging (fMRI) techniques are used to generate fMRI time series image data with respect to a selected regions of interest in the brain known to be affected by given disorders in response to activation tasks known to induce neural activity specifically in these regions. The fMRI data is used in generating statistically based standards and profiles by which abnormalities can be identified and the staging of disorders can be assessed. The results are diagrammatically or graphically displayed so that the data, standards and profiles can be usefully compared.

### RELATED APPLICATIONS

This application claims the benefit of U.S. provisional application No. 60/512,940 filed Oct. 21, 2003, which is incorporated herein by reference in its entirety.

### BACKGROUND OF THE INVENTION

This invention relates to medical imaging and the use of MRI machines and functional Magnetic Resonance Imaging (fMRI) techniques. More specifically, this invention relates to the use of fMRI techniques in detecting neural abnormalities for assisting in the diagnosis of CNS disorders and for assessing in gauging the staging of CNS disorders.

As the US population ages, disorders of the central nervous system (CNS) are becoming more common. Approximately 4 million Americans have Alzheimer's disease, with the number of cases expected to increase by 900,000 per year to over 13,000,000 by 2050. About 0.4% of the population over 40 is affected by Parkinson's disease and approximately 500,000 Americans suffer from Parkinson's disease, with about 50,000 new cases now expected to develop each year. The debilitating consequences of these and other CNS disorders, and their escalating prevalence, represent a challenge and opportunity for medical technologies that assist with diagnosis and treatment.

Currently, no technologies exist for the reliable early diagnosis of many CNS disorders and for the reliable assessment of therapies for use in treating such disorders. In particular, tests measuring the impact of the development of a disease on cognitive ability are lacking. Further, most clinical instruments have problems concerning reliability and sensitivity when they are used in assessing the modulation of symptoms as a function of therapeutic intervention. Early identification is important because new pharmacological, electrophysiological, surgical, and genetic treatments currently under development may prevent or delay disease onset. Early identification may allow the application of therapeutics before substantial degeneration has occurred preventing loss of cognitive ability, and delaying or preventing structural damage and mitigating symptoms. For example, no single test identifies Alzheimer's disease; physicians use a battery of physical and mental evaluations to identify a range of symptoms. While genetic testing for these diseases may offer indications that individuals are likely to develop symptoms, there are no predictors for timing of onset or for determining the severity of the disease. The early identification of many CNS diseases and early detection of the nature and the extent of such neurological changes on cognitive ability may be of great importance in the determination of therapy. For example, in Parkinson's disease the chronic use of L-DOPA therapy leads to a progressive diminution in its efficacy. Thus, it is desirable to monitor the progression of the disease more closely to effect possible changes in dosing. In the cases of most CNS disorders, quantitative measurement of the effects of therapies upon brain activity is very difficult at the present time.

Pharmaceutical companies developing therapeutic agents to treat CNS conditions generally rely on psychometric tools to evaluate a drug's impact on cognitive ability. Such tools are fraught with methodological imprecision, including low retest reliability, reduced sensitivity, and practice (learning) effects. As a consequence, an effective drug treatment may be inadvertently judged ineffective due to the imprecision of psychometric instruments. Furthermore, psychometric instruments are typically used to identify candidates for treatment. Because of their insensitivity, disease-related changes are detected years after the onset of pathological brain changes. As a result, drugs designed to prevent progression of the disease are instituted at a more advanced disease stage, resulting in reduced therapeutic efficacy. Currently, a large number of potential drugs targeted at treating CNS diseases are undergoing various stages of clinical trial evaluation. The requirements for clinical trials involve very high costs and can entail substantial delays in the approval process. Improved methods of identifying neural abnormalities, diagnosing and staging disease progression would reduce costs, help accelerate the development and approval process and increase the likelihood that therapeutic agents could be started earlier and with greater efficiency.

Anatomic Magnetic Resonance Imaging (MRI) is in widespread use to evaluate a wide variety of medical disorders, with total MRI services growing significantly each year. More than 15,000 MRI machines exist worldwide. MRI is now becoming a routine tool in the diagnosis of CNS disorders that produce observable structural abnormalities in the brain. These structural changes, such as brain atrophy, may occur late in the disease's progression and well after the onset of cognitive decline. Functional MRI, on the other hand, pinpoints changes in regional brain activity associated with cognitive, sensory, and motor tasks performed while the patient is being scanned. By using a rapid MR pulse sequence, a dynamic time-based sequence of MRI scans is acquired to detect hemodynamic changes reflecting neural activity. In its simplest form, fMRI is capable of detecting localized event related changes in MR signals over time. Its principal advantages over other non-invasive methods are its excellent spatial and temporal resolution and, as no isotopes are used, a virtually unlimited number of scanning sessions can be performed on a given subject, making within subject designs feasible. fMRI has the ability to detect increases in cerebral blood volume, flow, and oxygenation that locally occur in association with increased neuronal activity. A widely used fMRI method for following human brain activity is based upon blood oxygenation level dependent (BOLD) contrast. Although the physiological basis for BOLD signal fluctuations is not yet fully understood, it is proposed that neuronal activity leads to paradoxically increased levels of blood oxygenation, perhaps due to an excess increase of blood flow compared to increased oxygen consumption. Since oxyhemoglobin is diamagnetic while deoxyhemoglobin is paramagnetic, an increase in blood oxygenation results in increased field homogeneity (increase in T₂ and T₂*), less dephasing of the magnetic spins, and increased MR signal on susceptibility-weighted images.

Alternative imaging technologies, like positron emission tomography (PET), are capable of detecting functional brain activity, but with poorer spatial and temporal resolution than fMRI. Furthermore, PET involves the use of radioisotopes that limit its use in longitudinal research designs due to safety concerns, thereby eliminating its use in monitoring disease progression. In addition, functional brain imaging with PET requires short half-life isotopes, requiring that a cyclotron be present on-site adjacent to the PET scanner. As a consequence, only a limited number of medical centers have this capability.

### SUMMARY OF THE INVENTION

The present invention provides an fMRI system for addressing the assessment of neural abnormalities associated with central nervous system (CNS) disorders such as Alzheimer's Disease, Parkinson's Disease, Huntington's Disease, Multiple Sclerosis, Stroke, and Attention Deficit Hyperactivity Disorder and for assisting in conducting clinical trials to assess both the short and long-term effects and efficacy of different therapies and especially pharmaceuticals in treating CNS disorders. Whereas conventional MRI techniques and other imaging modalities focus on structural changes in the brain, the present invention employs fMRI to assess functional (cognitive, sensory, and motor) activity and detect functional changes associated with CNS diseases and the associated effects of therapeutic agents designed to treat CNS disorders. The system offers diagnostic and assessment tools for functional brain imaging that provide high levels of sensitivity and specificity and includes test procedures, data collection, and statistical analysis processes for efficiently processing, presenting and displaying fMRI data for use in diagnostic applications and for use in trials designed to assess drug efficacy.

fMRI imaging is applied in combination with particular stimuli and tasks that activate specific regions of the brain known to be affected by specific CNS disorders. Local brain activity is then measured on the basis of local hemodynamic changes (changes in deoxyhemoglobin concentration) that occur in response to the various activation tasks. Rapid T2*-sensitive imaging, usually gradient-echo echo-planar imaging, is performed during the performance of activation tasks and during rest periods. fMRI scans are collected and analyzed using techniques designed to extract signal intensity information from the time series collected. fMRI signal intensity information over time is correlated with the time course of the activation tasks to allow identification and visualization of task-related brain activity in the regions of interest. Comparisons may then be performed between the fMRI data for the images obtained during the stimulus task periods and during the rest periods. Patterns of neural activity uncovered in patients subject to CNS disorders can be compared to activity observed in healthy controls and with statistical norms for healthy individuals and also for patients known to be afflicted to varying degrees by particular CNS disorders. In this way neural abnormalities associated with CNS disorders may be identified in individual patients, disease progression and staging may be estimated and the affects of therapies assessed with reference to regions of the brain functionally affected by the pathology of particular disorders. The results can be presented as charts or graphical displays such as overlays on anatomic T1-weighted images of the patient's brain or activation maps which are illustrative of the condition of the patient relative to healthy individuals and other patients affected by the disorder or statistical plots which reference normal activation patterns characteristic of affected patients, patterns characteristic of affected patients subject to therapy or reference different patterns characteristic of the advancing stages in the progression of a disorder.

fMRI technology is advanced and extended to provide early diagnosis of numerous CNS disorders, where functional changes in brain activity may be exhibited years before structural changes can be seen on anatomical MRI scans or cognitive changes can be identified with psychometric instruments. Functional studies are designed to selectively activate one or more brain regions known to be affected by a CNS disorder using cognitive, sensory, or motor tasks. The resulting data are processed and subjected to statistical analyses with reference to fMRI databases containing baseline, disease progression and control data specific to the CNS disorder affecting the patient in order to identify neural abnormalities in support of diagnosing the disorder and assist in gauging the staging of the disorder.

It is an object of the present invention to provide a system for more rapidly and reliably identifying neural abnormalities in support of diagnosing CNS disorders, gauging the progression of CNS disorders and selecting appropriate therapies.

It is a further object of the present invention to provide a highly sensitive, noninvasive system using task-activated fMRI for use in identifying neural abnormalities in support of diagnosing CNS disorders, gauging the progression of CNS disorders and selecting optimum therapies.

It is another object of the present invention to provide a system using fMRI techniques for reliably detecting neural abnormalities and diagnosing CNS disorders which may be especially useful in detecting such disorders at pre-clinical and early clinical stages.

It is yet another object of the present invention to provide a system for collecting task-activated fMRI data and building a database of such data for use in defining the fMRI characteristics of the abnormalities associated with such CNS disorders, the staging of such disorders, the efficacy of therapies in treating such disorders and in developing statistically-based normative standards that address these characteristics.

### BRIEF DESCRIPTION OF THE DRAWINGS

FIG. 1 provides a diagrammatic illustration of a magnetic resonance imaging machine and its major components as adapted for performing functional magnetic resonance imaging studies.

FIG. 2 provides a diagrammatic illustration of the MRI system components specifically dedicated to the performance of functional magnetic resonance imaging studies in accordance with the present invention.

FIG. 3 provides a flowchart illustrating the operative process and steps for detecting and assessing neural abnormalities associated with a specific central nervous system disorders in accordance with the present invention.

FIG. 4 provides a diagram for a report generally illustrating how results pertaining to the detection and assessment of neural abnormalities may be usefully displayed in accordance with the present invention.

FIG. 5 provides a flowchart illustrating the operative process and steps for assessing the staging of a specific central nervous system disorder in individual patient suffering from the disorder in accordance with the present invention.

### DETAILED DESCRIPTION OF THE INVENTION

### I. fMRI Hardware

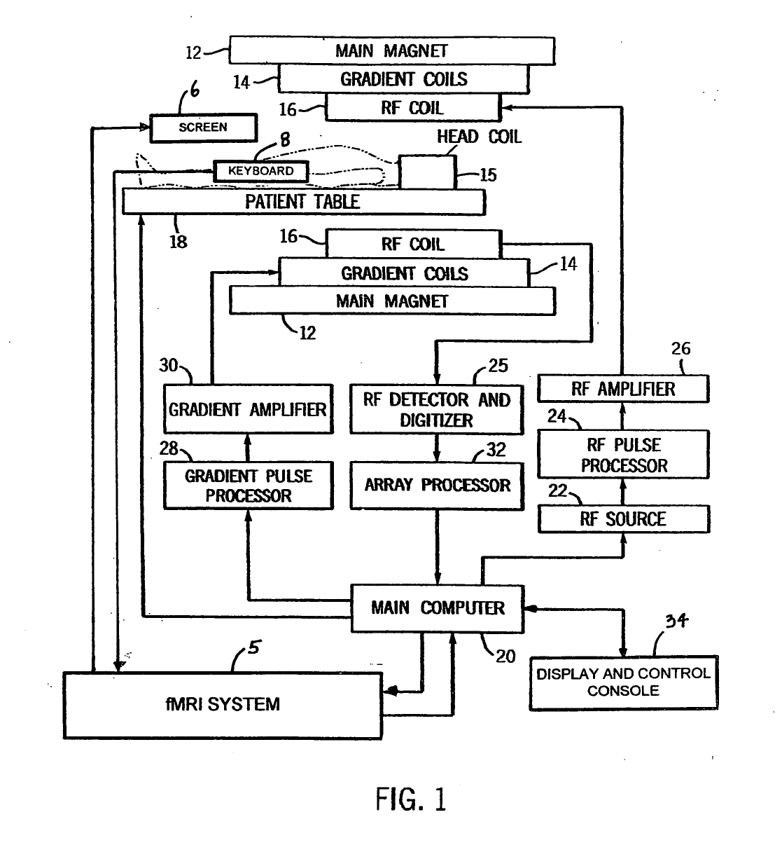

Referring now to FIG. 1, the basic components of a magnetic resonance imaging (MRI) machine 10 are shown including the fMRI system 5, which operates in conjunction with the MRI machine 10. A main magnet 12 produces a strong B₀ main magnetic field for use in the imaging procedure. Within the magnet 12 are the gradient coils 14 for producing a gradient in the B₀ field in the X, Y, and Z directions as necessary to provide frequency discrimination. A head coil 15 is also used to improve accuracy and resolution for studies involving the brain. Within the gradient coils 14 is a radio frequency (RF) coil 16 for producing RF pulses and the B₁ transverse magnetic field necessary to rotate magnetic spins by 90° or 180°. The RF coil 16 also detects the return signal from the spins within the body and supplies these signals to the RF detector and digitizer 25. The patient is positioned within the main magnet by a computer controlled patient table 18. The scan room is surrounded by an RF shield, which prevents the high power RF pulses from radiating out through the hospital and prevents the various RF signals from television and radio stations from being detected by the imager. The heart of the imager is the main MRI computer 20 that controls the components of the imaging system. The RF components under control of the computer include the radio frequency source 22 and pulse programmer 24. The source 22 produces a sine wave of the desired frequency. The pulse programmer 24 shapes the RF pulses into apodized sync pulses. The RF amplifier 26 greatly increases the power of the RF pulses. The computer 20 also controls the gradient pulse programmer 28 which sets the shape and amplitude of each of the three gradient fields. The gradient amplifier 30 increases the power of the gradient pulses to a level sufficient to drive the gradient coils 14. In most systems an array processor 32 is also provided for rapidly performing two-dimensional Fourier transforms. The MRI computer 20 off-loads Fourier transform tasks to this faster processing device. The operator of the imaging machine 10 provides input to the main MRI machine computer 20 through a display and control console 34. An imaging sequence is selected and customized by the operator from the console 34. The operator can see the MRI images on a video display located on the console 34. The fMRI system 5 controls the task display screen 6 visible to the subject and receives responses from the keyboard device 8 and coordinates the sequencing of activation task and MRI scanning procedures by exchanging signals with the main MRI computer 20.

Functional MRI is typically performed using a blipped, gradient-echo echo-planar (EP) pulse sequence with initial pi/2 pulse, TE of 40 ms [(kx,ky)=(0,0)], and 40 ms image acquisition time. Typical image resolution is 64×64 voxels with a 24 cm FOV, and 6 mm slice thickness (3.75×3.75×6 mm voxel size). Twenty-two contiguous sagittal slices are selected to provide coverage of the entire brain. For example, a General Electric Signa EXCITE 3.0 Tesla MRI scanner may be used for performing whole-brain imaging and implementing the present invention although any of a number of commercial MRI scanners having sufficiently intense (e.g. 3.0 or 1.5 Tesla) magnetic fields could be employed. Echo-planar (EP) images are typically collected using a single-shot, blipped, gradient echo EP pulse sequence; echo time (TE) =40 ms, with 40 ms of image acquisition time. The interscan period (TR) is about 2 seconds. Typical image resolution will be 64×64 voxels with a 24 cm field of view (FOV). Twenty-two contiguous sagittal 6 mm thick slices are selected in order to provide coverage of the entire brain (3.75×3.75×6 mm typical voxel size). An additional 6 images may be added to the beginning and end of the run to accommodate the delayed rise of the hemodynamic response. Prior to functional imaging, 124 high-resolution spoiled GRASS (gradient-recalled at steady-state) sagittal anatomic images [TE=5 ms; TR (repetition time)=24 ms, 40° flip angle, NEX (number of excitations)=1, slice thickness=1.2, FOV=24 cm, matrix size=256×128] are usually acquired on each subject. These images serve as the high-resolution anatomic images that allow precise localization of functional activity and co-registration. Foam padding is preferably used to limit head motion within the head coil. Head movement, typically subvoxel (<2 mm), is viewed in cine format. The image time series is spatially registered to minimize the effects of head motion and a 3D volume registration algorithm is used to align each volume in each time series to a fiducial volume through a gradient descent in a nonlinear least squares estimation of six movement parameters (3 shifts, 3 angles).

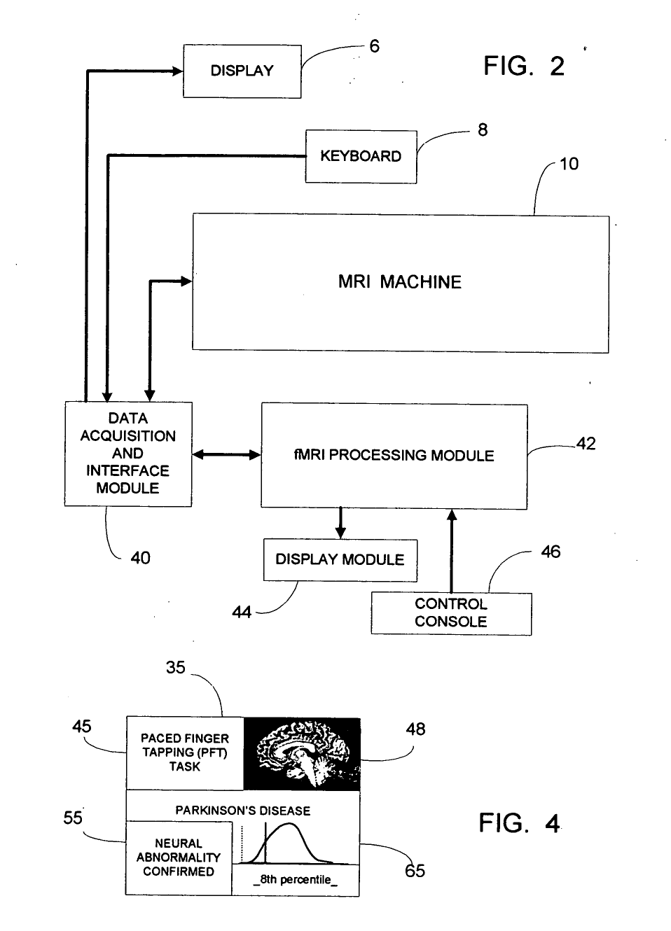

Referring now to FIG. 2, the fMRI system 5 includes the data acquisition and interface module 40, the processing module 42, the display module 44 and the input console 46 as well as the subject projection screen or display 6 and subject keyboard device 8. The module 40 directs the display of images to the subject on the screen 6 and also collects and preprocesses output responses from the subject provided from the keyboard device 8. The processing module 42 filters and analyses the fMRI data supplied to it by the data acquisition and interface module 40 by creating anatomical 3-dimensional datasets, converting the anatomical volumes into Talairach coordinate space, concatenating the functional time series datasets from multiple runs, registering the 3D time datasets to bring them into alignment, warping the functional datasets into Talairach coordinates, spatially blurring the images, performing deconvolution to compute the hemodynamic response to the stimuli, and calculating the change in hemodynamic response or BOLD contrast as a percent signal change over the region of interest (ROI). The processing module 44 also analyzes the data and compares the data with normative data, indices and standards derived from a normative database of data acquired under comparable conditions from large numbers of healthy subjects and patients afflicted with the same CNS disorders. The display module 44 displays the results The visual stimuli for the activation tasks are computer-generated by the fMRI system 5 and rear-projected (video projector) on an opaque screen 6 located in the vicinity of the subject's feet. The subjects view the screen through prism glasses attached to the head coil 15. Corrective lenses can be provided if necessary. The viewing distance is usually about 220 cm. A non-ferrous three-button key-press (keyboard) device 8 made from force-sensing resistors is preferably used to record responses, accuracy and reaction time. To provide precise time synchronization between the presentation of visual stimuli and the scan sequence, a trigger signal coincident with the acquisition of each MR image is fed into the computer controlled video display 6 by the fMRI system 5.

### II. fMRI Procedures and Operations

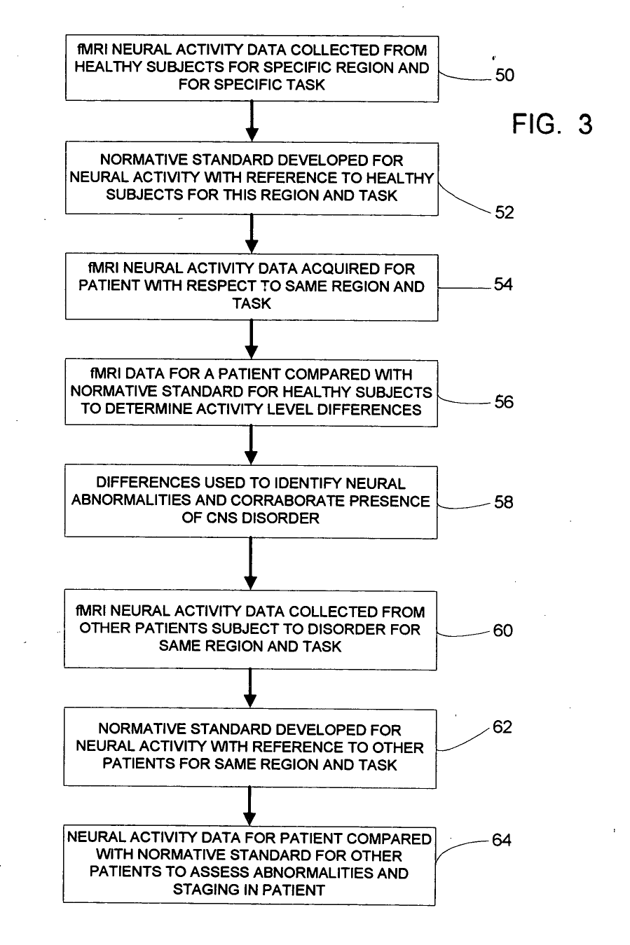

Referring now to FIG. 3, neural abnormalities associated with a CNS disorder may be assessed for an individual patient in support of diagnosing the disorder in accordance with steps 50-64 for processing fMRI data. In step 50 fMRI data indicative of neural activity from a large number of healthy subjects generated with respect to a specific region of interest in the brain known to be affected by the disorder and generated in response to a specific activation task known to induce functional activity in this region of the brain is collected. Thereafter, in step 52 a normative standard is developed for neural activity in the region of interest in response to the activation task based on statistical analysis of the fMRI data gathered from the healthy subjects. In step 54 fMRI data indicative of neural activity with respect to the specific region of interest in the brain and generated in response to the activation task is acquired from a patient having or suspected of having the disorder. In step 56 the neural activity indicated by fMRI data indicative of neural activity from the patient having or suspected of having the disorder is compared with the normative standard for neural activity by measuring one or more activity level differences between the activity for the patient and the normative standard. In step 58 neural abnormalities based on the one or more differences are identified and the presence of the disorder in the patient is corroborated 9 (or not corroborated) based on the differences in support of diagnosing the disorder in the patient. Thereafter, in step 60 fMRI data indicative of neural activity with respect to the specific region of interest in the brain and generated in response to the specific activation task is collected from other patients suffering from the disorder. In step 62 a standard is developed for altered neural activity in the region in response to the task in patients suffering from the disorder based on statistical analysis of the fMRI data gathered from the other patients. Finally, in step 64 the neural activity indicated by fMRI data indicative of neural activity from the patient having or suspected of having the disorder is compared with the normative standard for altered neural activity by measuring one or more activity level differences between the activity for the patient and the standard for altered activity in order to judge the extent and staging of the disorder.

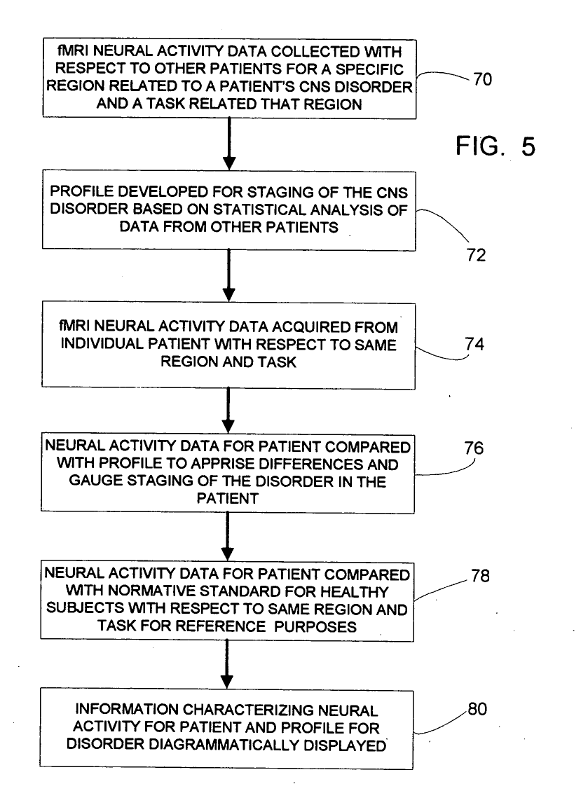

Referring now to FIG. 4, the reporting box 35 comprises a set of panels which visually display the essential elements of information comprising the results of an fMRI study with respect to identification of neural abnormalities or disorder staging. The activation task is identified in panel 45 and the region of interest is shown with reference to a brain map as panel 48. The region of interest would ordinarily be further identified by anatomical designation. The results are described in panel 55 and statistical information characterizing the result is graphically or diagrammatically shown in panel 65. The box 35 provides an efficient vehicle for concisely presenting the results of an fMRI study.

On this basis fMRI can be applied to the detection of neural abnormalities associated with central nervous system disorders. Neural abnormalities can be assessed on a functional basis and fMRI data can be used to quantify abnormalities on a patient-by-patient basis thereby facilitating the selection of optimum therapies and aiding in the determination of appropriate dosing for the application of medications. In particular, fMRI provides the ability to utilize quantitative measures of functional activity in the brain as a mechanism for evaluating disorders. Further, fMRI provides a mechanism for early diagnosis of CNS disorders, long before the structural damage has occurred in the brain that generates more visible symptomatic behavioral changes. By identifying functional changes caused by CNS disorder onset, fMRI can facilitate development of therapeutic strategies targeted toward the early prevention of structural damage.

Referring now to FIG. 5, the staging of CNS disorders may be evaluated for an individual patient and fMRI data relating to the staging of a disorder may be processed with respect to a specific region of interest in the brain known to be affected by the pathology of the disorder and a specific task known to induce activity in this region in accordance with steps 70-80. In step 70 fMRI data indicative of neural activity is collected from a large number of other patients suffering from different stages of the disorder generated with respect to a specific region of interest in the brain known to be affected by the disorder and generated in response to a specific activation task known to induce functional activity in this region of the brain. In step 72 a profile for altered neural activity according to the staging of the disorder in the region of interest in response to the activation task is generated based on statistical analysis of the fMRI data gathered from the patients. Thereafter, in step 74 fMRI data indicative of neural activity with respect to the specific region of interest in the brain and generated in response to the activation task is acquired from an individual patient suffering from the disorder. In step 76 fMRI data indicative of neural activity from the patient suffering from the disorder generated with respect to the region of the brain and generated in response to the activation task is compared with the profile of neural activity by apprising one or more differences between the neural activity for the patient and the profile of the disease. The stage of the disorder in the patient is gauged based on these differences. Thereafter, in step 78 the neural activity indicated by the fMRI data indicative of neural activity from the patient suffering from the disorder is compared with a normative standard for neural activity with respect to the specific region of interest in the brain and generated in response to the specific activation task developed by statistical analysis of fMRI data from a large population of healthy subjects in order to apprise differences between the activity for the patient and the normative standard and to better understand the scale of the patient's abnormalities in relation to healthy subjects. Finally in step 80 the neural activity of the patient and the profile for altered neural activity according to the staging of the disorder are diagrammatically displayed in conjunction with information identifying the region of interest and the activation task in order to provide a concise visually instructive output and a comparative evaluation of the activity and profile.

Normative databases are important for use in analyzing and maintaining fMRI data as it pertains to identifying abnormalities in support of diagnosing CNS disorders and their symptoms, and as it relates to assessing and monitoring the efficacy of pharmacological therapies that target these disorders. To be most effective, these databases need to extend over large populations and include data from individuals of both sexes and a wide range of ethnic, age, education and health backgrounds. With large data samples and standardized techniques more accurate and reliable results can be achieved especially in addressing the early stages of various CNS disorders. The database also provides a statistical framework for reliably analyzing fMRI data and achieving accurate results. The normative database is dynamic and improves as more data are collected and as more comparative studies of populations measured against various regions and tasks are completed and merged into the database.

In practice, these databases allow for lookup tables for use in the field to be generated based on compiled data which express the statistical ranges for normal performance data and allow individual sets of fMRI data to be accurately placed with respect to normative standards for levels and spatial extent of neural activity and with respect to profiles featuring varying activity levels according to disease state established with reference to large populations of healthy individuals and large groups of patients suffering from CNS disorders. These databases serve to manage patient and population data for research purposes and in support of the commercial application of fMRI in diagnosis, disorder staging, medication dosing and therapy evaluation. As new data are received the databases allow updates to be generated for disorder and therapy assessment modules in order to assure that the latest statistical information on patient populations is provided for use in the field.

Once sufficient fMRI image data is developed with respect to particular CNS disorders regions of interest and activation tasks fMRI can be further utilized as a tool to help identify and establish new fMRI based biomarkers. Based on preliminary studies cognitive or physiological activation tasks are designed that activate localized regions of the brain that are known to be affected by the pathology of a particular disease. Further fMRI data and experience may then be used to highlight a pattern of change from normal signal levels specific to the CNS disease and demonstrate a clear statistical connection between this pattern with respect to the BOLD signal and the pathology of the disorder in order to advance the validation of the task and region as a biomarker. A particular disease marker is provided by a unique localized area (or pattern of areas) in the brain in which there are reliable changes in the relative level of the BOLD signal response between normal and disorder afflicted populations as a function of the pathology of the disorder. Specific cognitive or physiological tasks may stimulate unique responses in the relative BOLD signal intensity or spatial extent in localized regions in the brain. As the pathology of a particular disease causes damage to particular foci in the brain, these areas may show selective increases or decreases in BOLD signal intensity in comparison to healthy individuals. In particular with decreased levels of activity in one part of the brain, there may be increased levels of activity in other brain regions as the brain attempts to compensate by recruiting other areas to fill in for lost functionality. These changes in patterns of neural activation for different disorders, regions and tasks are believed to be common in populations afflicted by CNS disorders. fMRI biomarkers are integral to the establishment of normative databases and integral in using fMRI in addressing CNS disorders. As more studies are performed and as more clinical information is collected, the specific biomarkers for specific CNS disorders are continually refined to provide more reliable and sensitive determinations that further complement the understanding of these disorders and improve the effectiveness of fMRI techniques.

### III. Operation Examples

### Time Discrimination in Presymptomatic Huntington's Disease

Task-activated functional MRI (fMRI) was used as a probe of basal ganglia function in pre-symptomatic Huntington's Disease (pre-HD). A previous fMRI study conducted in healthy individuals demonstrated activation of the basal ganglia, and particularly the caudate, during a time discrimination study. The current study was designed to examine the relative sensitivity of fMRI in detecting early HD-related neurodegenerative changes in comparison to behavioral testing and morphometric measurements derived from structural MRI. Fourteen Pre-HD participants were subdivided into two groups based upon estimated years to diagnosis (seven CLOSE=<12 years; seven FAR=>12 years). The participants were matched by age and education to seven controls. Disease onset age was estimated using a regression equation based on the number of CAG repeats and the affected parent's age of onset. The time discrimination task required participants to determine whether a specified interval was longer or shorter than a standard interval (1200 ms). For this study event-related fMRI was performed on a 1.5 Tesla General Electric Signa scanner equipped with a 3-axis local gradient head coil and an elliptical end-capped quadrature radiofrequency coil. Foam padding was used to limit head motion within the coil. Prior to functional imaging, high-resolution, three-dimensional (3D), spoiled gradient-recalled at steady-state images were collected (TE=5 ms, TR=24 ms, 40° flip angle, NEX=1, slice thickness=1.2 mm, FOV=24 cm, matrix=256×128) for anatomic localization and co-registration. For functional imaging, echo-planar images were collected using a single-shot, blipped gradient-echo echo-planar pulse sequence (TE=40 ms, TR=2.5 seconds, 90° flip angle, FOV=24 cm, resolution=64×64 matrix). Nineteen contiguous sagittal 7 mm thick slices were collected to provide coverage of the entire brain. Scanning was synchronized with the onset of the activation task so that the 7 images were acquired during each trial. There were 16 trials per run. An additional 4 images were added at the start of each run to allow the MR signal to reach equilibrium and 4 images were added at the end to accommodate hemodynamic response delay. Overall, a total of 120 images were collected per run.

CLOSE participants performed more poorly on the time discrimination than controls; however, no perceptual differences were observed between FAR participants and controls. Similarly, CLOSE participants demonstrated a reduction in volume of the caudate bilaterally relative to controls, whereas FAR participants did not. On functional imaging, CLOSE participants displayed significantly less activation in subcortical regions (caudate, thalamus) than controls, whereas FAR participants demonstrated an intermediate degree of activation. In contrast, FAR participants displayed hyperactivation in medial wall structures (anterior cingulate, supplementary motor area) relative to CLOSE and control participants. Hyperactivation of medial prefrontal regions compensated for reduced subcortical participation during time discrimination in early HD. This pattern of brain activation may represent an early neurobiological indication and marker of disease progression.

### Neural Basis for Impaired Time Reproduction in Parkinson's Disease

Patients with Parkinson's disease (PD), for example, demonstrate abnormal performance on the paced finger tapping (PFT), characterized by decreased accuracy and variability changes, suggesting that the basal ganglia may play a critical role in motor timing. Consistent with this hypothesis, an fMRI study of healthy participants demonstrated that the medial frontostriatal circuit (dorsal putamen, ventrolateral thalamus, SMA) correlated with explicit time-dependent components of the PFT task. In this fMRI study, ten PD patients and thirteen healthy age-matched controls were imaged while performing the PFT. PD patients underwent two imaging sessions, one on and the other off dopamine supplementation. For this study whole-brain fMRI was performed on a 1.5 Tesla General Electric Signa scanner equipped with a 3-axis local gradient head coil and an elliptical end-capped quadrature radiofrequency coil. Foam padding was used to limit head motion within the coil. Echo-planar images were collected using a single-shot, blipped gradient-echo echo-planar pulse sequence (TE=40 ms, TR=3.0 seconds, 90° flip angle, FOV=240 mm, matrix=64×64). Twenty-two contiguous sagittal 6 mm thick slices were collected to provide coverage of the entire brain (voxel size 3.75×3.75×6 mm). Overall, a total of 136 images were collected per run. Prior to functional imaging, high-resolution, three-dimensional, spoiled gradient-recalled at steady-state (GRASS) anatomic images were collected (TE=5 ms, TR=24 ms, 40° flip angle, NEX=1, slice thickness=1.2 mm, FOV=24 cm, matrix=256×128) for anatomic localization and co-registration.

Relative to controls, PD patients were less accurate and showed greater variability on the PFT task relative to controls. No PFT performance differences were observed between the on and off medication states despite significantly greater motor symptoms on the Unified Parkinson's Disease Rating Scale (UPDRS) in the off medication state. Functional imaging results demonstrated decreased activation within the sensorimotor cortex (SMC), cerebellum, and medial premotor system in the PD patients compared to controls. With dopamine replacement, an increase in the spatial extent of activation was observed within the SMC, SMA, and putamen in the PD patients. These results indicate that impaired timing reproduction in PD patients is associated with reduced brain activation within motor and medial premotor circuits. Despite a lack of improvement in PFT performance, PD patient's brain activation patterns were partially “normalized” with dopamine supplementation. As expected from our previous fMRI study conducted with healthy young subjects, robust activation of the left sensorimotor cortex (SMC), bilateral superior temporal gyrus (STG), and right cerebellum was observed during the S condition. The PD patients, either ON or OFF medication, showed a reduced spatial extent of activation relative to the Controls, with the only region of overlap occurring in the STG. In the SMC, the area of activation for the PD group (ON and OFF) was located more caudal to that observed in the Control subjects. Levodopa had relatively little effect on the S task. Activation of the putamen and thalamus, however, was observed in the PD patients when they were ON, but not OFF, medication. The SMA was not activated in the PD patients while OFF medication; however, activation was observed during the ON state, albeit smaller in spatial extent than the Controls. In addition, the PD patients ON medication had more rostral activation of the SMC, similar to the maps of the Control subjects and unlike that of the patients when OFF medication. Unlike the Controls, no activation was observed in the right cerebellum of the PD patients either ON or OFF medication.

Although the invention has been described with reference to certain embodiments for which many implementation details have been described, it should be recognized that there are other embodiments within the spirit and scope of the claims and the invention is not intended to be limited to the details described with respect to the embodiments disclosed.

1) A method of detecting neural abnormalities associated with a specific central nervous system disorder in support of diagnosing said disorder in patients suffering from or suspected of suffering from this disorder, comprising the steps of:
a) collecting fMRI data indicative of neural activity from a large number of healthy subjects generated with respect to a specific region of interest in the brain known to be affected by the disorder and generated in response to a specific activation task known to induce functional activity in this region of the brain;
b) developing a normative standard for neural activity in said region of interest in response to said activation task based on statistical analysis of said fMRI data gathered from said healthy subjects;
c) acquiring fMRI data indicative of neural activity with respect to said specific region of interest in the brain and generated in response to said activation task from a patient having or suspected of having the disorder;
d) comparing the neural activity indicated by fMRI data indicative of neural activity from said patient having or suspected of having said disorder with said normative standard for neural activity by measuring one or more activity level differences between said activity for said patient and said normative standard; and
e) identifying neural abnormalities based on said one or more differences.

2) The method of claim 1, further including the step of:
corroborating the presence of said disorder in said patient based on said differences.

3) The method of claim 1, in which:
said step of collecting fMRI data includes the step of forming a normative database including fMRI data from a large population of subjects.

4) The method of claim 1, in which:
said neural activity is characterized in terms of percent changes in signal level from rest active to control state.

5) The method of claim 1, further including the steps of:
collecting fMRI data indicative of neural activity with respect to said specific region of interest in the brain and generated in response to said specific activation task from other patients suffering from the disorder,
developing a standard for altered neural activity in said region in response to said task in patients suffering from the disorder based on statistical analysis of said fMRI data gathered from said other patients,
comparing the neural activity indicated by fMRI data indicative of neural activity from said patient having or suspected of having said disorder with said normative standard for altered neural activity by measuring one or more activity level differences between said activity for said patient and said standard for altered activity.

6) A method of assessing the staging of a specific central nervous system disorder in individual patient suffering from this disorder, comprising the steps of:
a) collecting fMRI data indicative of neural activity from a large number of other patients having different stages of said disorder generated with respect to a specific region of interest in the brain known to be affected by the disorder and generated in response to a specific activation task known to induce functional activity in this region of the brain;
b) generating a profile for altered neural activity according to the staging of the disorder in said region of interest in response to said activation task based on statistical analysis of said fMRI data gathered from said from said other patients;
c) acquiring fMRI data indicative of neural activity with respect to said specific region of interest in the brain and generated in response to said activation task from a patient suffering from the disorder;
d) comparing the neural activity indicated by fMRI data indicative of neural activity from said patient suffering from said disorder generated with respect to said region of the brain and generated in response to said activation task with said profile of neural activity by apprising one or more differences between said activity for said patient and said profile; and
e) gauging the stage of said disorder in said patient based on said differences.

7) The method of claim 6, further including the step of:
recommending a therapy for said patient based on said differences in neural activity.

8) The method of claim 6, further including the step of:
said step of collecting fMRI data includes the step of forming a normative database including fMRI data from a large population of other patients.

9) The method of claim 6, in which:
said neural activity is characterized in terms of percent changes in signal level from rest active to control state.

10) The method of claim 6, further including the step of:
comparing the neural activity indicated by fMRI data indicative of neural activity from said patient suffering from said disorder with a normative standard for neural activity with respect to said specific region of interest in the brain and generated in response to said specific activation task developed by statistical analysis of fMRI data from a large population of healthy subjects to apprise differences between said activity for said patient and said normative standard.

11) The method of claim 6, further including the step of:
diagrammatically displaying the neurological activity of said patient and said profile for altered neural activity according to the staging of the disorder in conjunction with information identifying said region of interest and said activation task.

12) A system for detecting neural abnormalities associated with a specific central nervous system disorder in support of diagnosing said disorder in patients suffering from or suspected of suffering from this disorder and assessing the staging of said disorder in patients suffering from or suspected of having the disorder using an MRI machine and fMRI techniques in which fMRI data is generated with respect to a selected region of interest in the brain known to be affected by the disorder in response to an activation task known to induce neural activity specifically in this region, said system comprising the steps of:
a) generating a normative standard for levels of neural activity in said region of interest in response to said activation task in healthy individuals by statistical analysis of fMRI data from healthy subjects generated with respect to said region of interest and in response to said activation task;
b) acquiring fMRI data indicative of levels of neural activity with respect to said specific region of interest in the brain and generated in response to a specific activation task from an individual patient suffering from the disorder;
c) comparing said levels of neural activity indicated by said fMRI data for said individual patient with said normative standard to apprise any differences and identify neural abnormalities indicative of said disorder; and
d) diagrammatically displaying said level of neural activity in said patient with reference to said normative standard in conjunction with information identifying said region of interest and said task.

13) The method of claim 12, further including the steps of:
generating a profile for altered neural activity according to the staging of the disorder in said region of interest in response to said activation task based on statistical analysis of fMRI data from patients generated with respect to said region of interest and in response to said activation task,
comparing the neural activity indicated by said fMRI data for said individual patient with said profile to apprise any differences indicative of the staging of said disorder.

14) The method of claim 12, in which:
said normative standard for neural activity in said region in response to said task in healthy subjects is derived by statistical analysis of fMRI data in a normative database including data gathered from a large population of subjects.

15) The method of claim 12, in which:
said neural activity is characterized in terms of percent changes in signal level from active to control state.
